(19) United States
(12) Patent Application Publication
(10) Pub. No.: US 2015/0366530 A1
(43) Pub. Date: Dec. 24, 2015

(54) DEVICE, COMPUTING DEVICE AND METHOD FOR DETECTING FISTULA STENOSIS

(71) Applicant: MediaTek Inc., Hsin-Chu (TW)

(72) Inventor: Po-Wen KU, Jhubei City (TW)

(21) Appl. No.: 14/312,916

(22) Filed: Jun. 24, 2014

(51) Int. Cl. A61B 7/04; A61B 5/00; A61B 5/02; A61M 1/36; A61B 5/0402

(52) U.S. Cl. CPC A61B 7/04; A61M 1/3658; A61B 5/04028; A61B 5/02; A61B 5/7282; A61M 2230/04; A61M 2210/12

(57) ABSTRACT

A device for detecting fistula stenosis is provided. The device includes a physiological signal sensor, an acoustic receiver and a processing circuit. The physiological signal sensor is configured for providing a physiological signal of a user. The acoustic receiver is configured for detecting a sound from a fistula of the user to generate a sound signal. The processing circuit is configured for providing a degree of fistula stenosis according to the physiological signal of the user and the sound signal.

### BACKGROUND OF THE INVENTION

1. Field of the Invention

The invention relates to a device for detecting fistula state, and more particularly to a device for detecting fistula stenosis.

2. Description of the Related Art

For dialysis patients, fistula is widely used in the metabolism of waste removed via a dialysis machine and it is very important to keep fistula functioning normally. Fistula stenosis is a major cause of dysfunction of fistula and poses very serious threats for dialysis patients. As blood of dialysis patients flows through a fistula, the diameter of the fistula may be gradually reduced. When the diameter of fistula is reduced to only around 50% of its original scale, it may be considered to have fistula stenosis. Detecting the degree of fistula stenosis is one effective approach to avoid fistula stenosis. In general, the degree of fistula stenosis is determined by collecting and analyzing sound generated when blood stream passes through the fistula.

Therefore, an accurate device is necessary to monitor the fistula for resident care, so as to effectively detect fistula stenosis.

### BRIEF SUMMARY OF THE INVENTION

Device, computing device, method and computer readable storage medium for detecting fistula stenosis are provided. An embodiment of a device for detecting fistula stenosis is provided. The device comprises: a physiological signal sensor, configured for providing a physiological signal of a user; an acoustic receiver, configured for detecting a sound from a fistula of the user to generate a sound signal; and a processing circuit, configured for providing a degree of fistula stenosis according to the physiological signal of the user and the sound signal.

Furthermore, an embodiment of a computing device for detecting fistula stenosis is provided. The computing device comprises a processing circuit. The processing circuit comprises a processor, configured for providing a degree of fistula stenosis according to a physiological signal and a sound signal.

Moreover, a method for detecting fistula stenosis is provided. At a computing device, a physiological signal of a user is received, wherein the physiological signal is provided by a physiological signal sensor. At the computing device, a sound signal generated by an acoustic receiver is received, wherein the acoustic receiver detects a sound from a fistula of the user to generate the sound signal. At the computing device, a degree of fistula stenosis is provided according to the physiological signal of the user and the sound signal.

In another embodiment, a computer readable storage medium having stored therein instructions is provided. The instructions, when executed by a device, cause the device to provide a degree of fistula stenosis according to a physiological signal of a user and a sound signal.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 shows a device for detecting fistula stenosis of a user according to an embodiment of the invention;

FIG. 2 shows a method for detecting fistula stenosis of a user according to an embodiment of the invention;

FIG. 3A shows an example illustrating the relationship between a physiological signal S_{ECG} and a fistula sound signal S_{FISTULA} according to an embodiment of the invention;

FIG. 3B shows an example illustrating the relationship between a physiological signal S_{PPG} and a fistula sound signal S_{FISTULA} according to an embodiment of the invention;

FIG. 4 shows a device for detecting fistula stenosis of a user according to another embodiment of the invention;

FIG. 5 shows an example model illustrating a device for detecting fistula stenosis of a user according to an embodiment of the invention;

FIG. 6 shows a schematic illustrating using the device of FIG. 5 to measure the fistula of the user;

FIG. 7 shows an example illustrating the relationship between another physiological signal S_{ECG}_{_{—}}₁ and a heart sound signal S_{HEART} according to an embodiment of the invention; and

FIG. 8 shows an example model illustrating a device 800 for detecting fistula stenosis of a user according to another embodiment of the invention.

### DETAILED DESCRIPTION OF THE INVENTION

The following description is made for the purpose of illustrating the general principles of the invention and should not be taken in a limiting sense. The scope of the invention is best determined by reference to the appended claims.

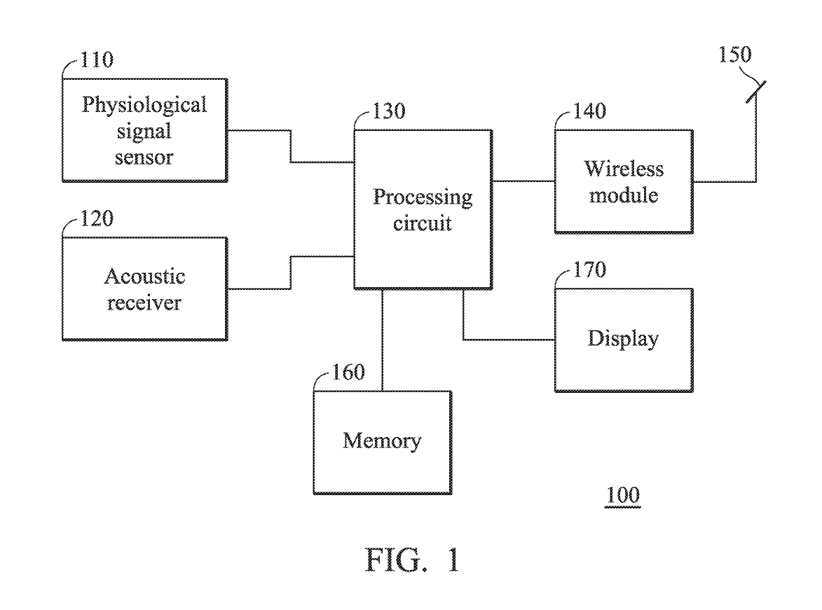

FIG. 1 shows a device 100 for detecting fistula stenosis of a user according to an embodiment of the invention. The device 100 comprises a physiological signal sensor 110, an acoustic receiver 120, a processing circuit 130, a wireless module 140, an antenna 150, a memory 160 and a display 170. The wireless module 140 is capable of communicating with a remote device via the antenna 150. When the device 100 properly contacts the skin of a user near a fistula of the user, the physiological signal sensor 110 is capable of providing a physiological signal of the user. Simultaneously, the acoustic receiver 120 is capable of detecting a sound from the fistula of the user to generate a sound signal. Next, the processing circuit 130 derives or provides a degree of fistula stenosis according to the physiological signal of the user and the sound signal. For example, firstly, the processing circuit 130 processes the physiological signal to derive a physiological information, which identifies the diastolic phases and systolic phases of cardiac cycles of the user. Secondly, the processing circuit 130 partitions the sound signal into a plurality of intervals according to the physiological information. For an interval of the sound signal, it either corresponds to a diastolic phase of a cardiac cycle of the user or a systolic phase of a cardiac cycle of the user. To be specific, a diastolic phase of a cardiac cycle may span from t₀ ms to t₀+t_{d} ms and a systolic phase of the cardiac cycle may span from t₀+t_{d} ms to t₀+t_{d}+tₛ ms. Then, for an interval of the sound signal corresponding to the diastolic phase of the cardiac cycle, it may mean the interval of the sound signal spans from t₀ ms to t₀+t_{d} ms. Similarly, for an interval of the sound signal corresponding to the systolic phase of the cardiac cycle, it may mean the interval of the sound signal spans from t₀+t_{d} ms to t₀+t_{d}+tₛ ms. Thirdly, the processing circuit 130 may use different algorithms, databases or approaches to analyze an interval of the sound signal corresponding to a diastolic phase of a cardiac cycle and another interval of the sound signal corresponding to a systolic phase of a cardiac cycle, so as to determine the degree of fistula stenosis. This is because the audio characteristics of a sound signal during a diastolic phase may be clearly different from the audio characteristics of a sound signal during a systolic phase.

In one embodiment, the processing circuit 130 may have access to a first database storing a frequency response of a sound signal obtained from a fistula with fistula stenosis during a diastolic phase of a cardiac cycle. The processing circuit 130 may also have access to a second database storing a frequency response of a sound signal obtained from a fistula with fistula stenosis during a systolic phase of a cardiac cycle. For a first interval of the sound signal corresponding to a diastolic phase, the processor 130 may derive a first frequency response of the first interval of the sound signal, and then the processing circuit 130 compares the first frequency response with the frequency response stored in the first database to determine the degree of fistula stenosis. For a second interval of the sound signal corresponding to a systolic phase, the processing circuit 130 may derive a second frequency response of the second interval of the sound signal, and then the processing circuit 130 compares the second frequency response with the frequency response stored in the second database. If the processing circuit 130 finds the first frequency response to be quite similar to the frequency response stored in the first database and the second frequency response to be quite similar to the frequency response stored in the second database, the degree or level of fistula stenosis may be declared to be high by setting an index to a corresponding value. For instance, the index may be a positive integer from 1 to 10. When the index is set to 1, it means stenosis is less than 10% of the fistula. On the other hand, when the index is set to 10, it means stenosis is greater than 90% but smaller than 100% of the fistula. If the processing circuit 130 finds the first frequency response to be quite different from the frequency response stored in the first database and the second frequency response to be quite different from the frequency response stored in the second database, the degree or level of fistula stenosis may be declared to be low and the index above may be set to near 1.

To elaborate more, for a frequency response of a sound signal during a systolic phase of a cardiac cycle, experimental result shows signal energy above, say, 100 Hz increases as the degree of fistula stenosis increases. However, for a frequency response of a sound signal during a diastolic phase of a cardiac cycle, signal energy above, say, 100 Hz remains relatively similar as fistula stenosis increases. This prompts one to distinguish the sound signal according to diastolic and systolic phases when determining the degree of fistula stenosis. For example, one may analyze a portion of the sound signal corresponding to diastolic phases and another portion of the sound signal corresponding to systolic phases separately with different criterion. Specifically speaking, for a first portion of the sound signal corresponding to systolic phases of cardiac cycles of the user and a second portion of the sound signal corresponding to diastolic phases of cardiac cycles of the user, the second portion of the sound signal is bypassed or ignored from analysis and the first portion of the sound signal is analyzed to derive the degree of fistula stenosis.

Note that, there are other techniques such as machine learning that can be combined or used for determining the degree of fistula stenosis not departing from the spirit of distinguishing a first portion of the sound signal corresponding to diastolic phases of cardiac cycles and a second portion of the sound signal corresponding to systolic phases of cardiac cycles, and they shall all fall within the scope of this invention. Furthermore, the processing circuit 130 may store the audio characteristics of the sound signal into the memory 160. In FIG. 1, the physiological signal may be an electrocardiogram (ECG) lead signal or a photoplethysmography (PPG) signal. Also, the degree of fistula stenosis can be displayed via the display 170 and the degree of fistula stenosis, the sound signal or the physiological signal can be transmitted to a remote device for further medical analysis. As another example, to inform a user the degree of fistula stenosis, an audio signal may be generated instead of using a visual signal to show the degree of fistula stenosis. Then, the display 170 may be replaced by a speaker, which serves as an output unit. In addition, the device 100 may be a portable device and fistula of the user may be one of an autogenous arteriovenous fistula and an arteriovenous fistula graft.

It has to be pointed out that the processing circuit 130 may be a general purpose processor or a digital signal processor that receives a specific instruction set to execute tasks; however, the processing circuit 130 may also be a dedicate hardware or implemented in application specific integrated circuit (ASIC). To provide the degree of fistula stenosis according to the physiological signal of the user and the sound signal, the processing circuit 130 may have an information generator, a signal separator, and a signal analyzer. The information generator derives the physiological information according to the physiological signal of the user. A physiological information such as a diastolic phase or a systolic phase of a cardiac cycle may be obtained by observing the time domain waveform of an ECG lead signal. The signal separator then distinguishes a first portion of the sound signal from a second portion of the sound signal according to the physiological information; in other words, there may be two signal paths after the signal separator. One signal path has a first portion of the sound signal and the other signal path has a second portion of the sound signal. The first portion corresponds to diastolic phases of cardiac cycles and the second portion corresponds to systolic phases of cardiac cycles. Then, the signal analyzer analyzes at least one of the first portion of the sound signal and the second portion of the sound signal to derive the degree of fistula stenosis. As an example, the signal analyzer analyzes the second portion of the sound signal and ignores the first portion of the sound signal so as to get more accurate degree of fistula stenosis.

When the processing circuit 130 is a processor, the processor may execute instructions to provide a degree of fistula stenosis according to a physiological signal and a sound signal. In other words, similar tasks performed by the information generator, the signal separator and the signal analyzer above may also be performed by the processor receiving adequate instructions. Note that the instructions executed by the processor may be provided in the form of an application program. When a user wants to know the degree of fistula stenosis, the application program may be downloaded from a computer readable storage medium such as an internet disk drive, a cloud storage, or an optical disk to the processor. When the application program is run on the processor, the processor executes the instructions to provide a degree of fistula stenosis according to a physiological signal of a user and a sound signal.

It may be possible that the physiological signal sensor 110, the acoustic receiver 120 and the processing circuit 130 are separate components. For example, the processing circuit 130 may be inside a computing device and the physiological signal sensor 110 and the acoustic receiver 120 may be dongled to the computing device. That is, when needed, the physiological signal sensor 110 and the acoustic receiver 120 are attached to the computing device such that the processing circuit 130 can receive a physiological signal and a sound signal for deriving the degree of fistula stenosis.

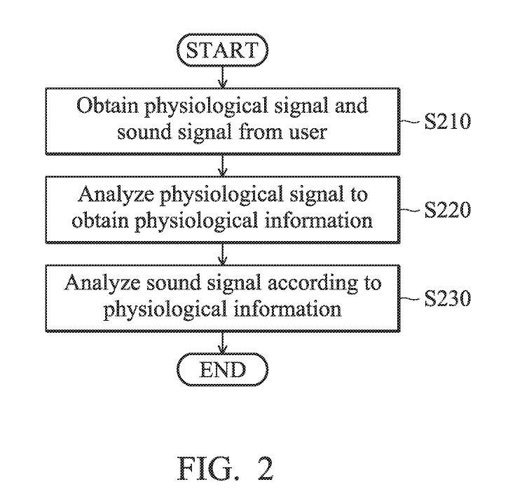

FIG. 2 shows a method for detecting fistula stenosis of an user according to an embodiment of the invention. Referring to FIG. 1 and FIG. 2 together, first, in step S210, the processing circuit 130 obtains a physiological signal of the user via the physiological signal sensor 110 and a sound signal from a fistula of the user via the acoustic receiver 120 when the device 100 properly contacts the user. Next, in step S220, the processing circuit 130 analyzes the physiological signal to obtain physiological information of the user. In one embodiment, the physiological information identifies intervals of a diastolic phase and/or a systolic phase of at least one cardiac cycle of the user. In another embodiment, the physiological information identifies intervals of an arrhythmia duration and/or a non-arrhythmia duration of a plurality of cardiac cycles of the user. Next, in step S230, the processing circuit 130 analyzes the sound signal according to the physiological information. For example, an interval of the sound signal may correspond to a diastolic phase of the cardiac cycle, a systolic phase of the cardiac cycle, arrhythmia duration of the cardiac cycles, or non-arrhythmia duration of the cardiac cycles. The processing circuit 130 then chooses a suitable algorithm to analyze different intervals of the sound signal according to the physiological information. The reason to choose a suitable algorithm is because, for example, the audio characteristics of a sound signal during arrhythmia duration may be clearly different from the audio characteristics of a sound signal during non-arrhythmia duration.

Note that the audio characteristics may be a frequency response, signal strength or any informative attributes of the interval of the sound signal. For example, the processing circuit 130 can use a first algorithm to analyze the interval of the sound signal corresponding to the diastolic phase of the cardiac cycle, and use a second algorithm to analyze the interval of the sound signal corresponding to the systolic phase of the cardiac cycle. Compared to the second algorithm, the first algorithm may use iterative approach with parameters having larger convergence speed since during diastolic phase the sound signal may vary more fastly.

In one embodiment, the processing circuit 130 can use a third algorithm to analyze the interval of the sound signal corresponding to the arrhythmia duration of the cardiac cycles, and use a fourth algorithm to analyze the interval of the sound signal corresponding to the non-arrhythmia duration of the cardiac cycles. For instance, a first signal strength of an interval of the sound signal corresponding to a diastolic phase of a cardiac cycle is derived and a second signal strength of another interval of the sound signal corresponding to a systolic phase of a cardiac cycle is also derived. The processing circuit 130 then compares the first signal strength with a first threshold and compares the second signal strength with a second threshold. If the first signal strength exceeds the first threshold and the second signal strength exceeds the second threshold, it may be judged that the degree of fistula stenosis is low. On the contrary, if the first signal strength is smaller than the first threshold and the second signal strength is smaller than the second threshold, it may be judged that the degree of fistula stenosis is high. This is because as degree of fistula stenosis increases, the magnitude of sound signal generated while blood stream passes through the fistula may be reduced. In another embodiment, the processing circuit 130 analyzes the interval of the sound signal corresponding to the non-arrhythmia duration of the cardiac cycles and not analyzes the interval of the sound signal corresponding to the arrhythmia duration of the cardiac cycles so as to determine the degree of fistula stenosis. This is because during the arrhythmia duration of the cardiac cycles, the fistula sound signal may be not reliable enough for making a good judgment of fistula stenosis. In other words, the portion of the sound signal corresponding to arrhythmia duration is ignored or bypassed when determining the degree of fistula stenosis.

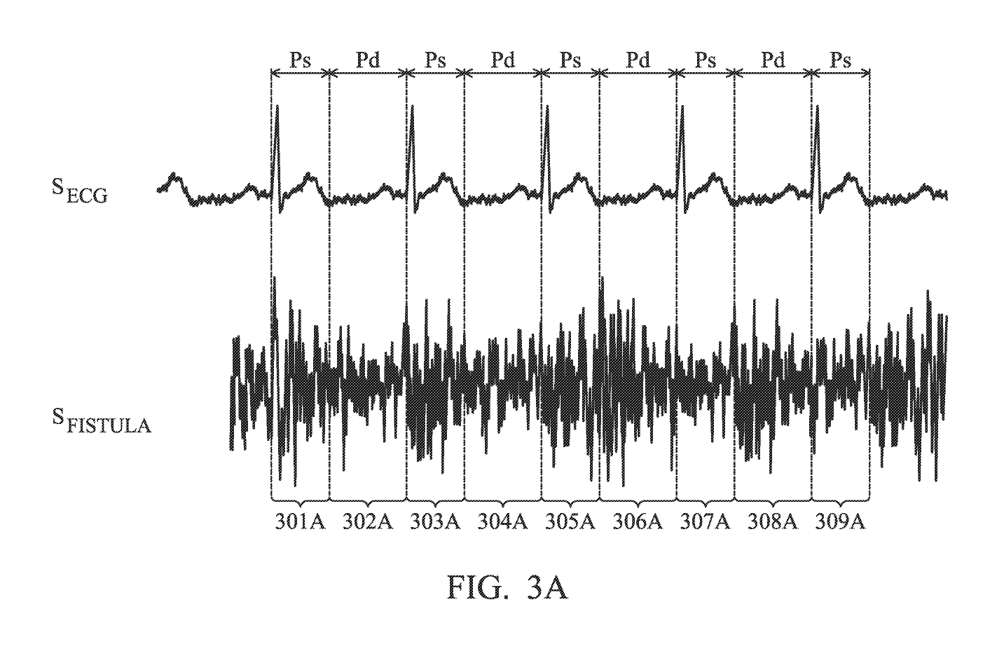

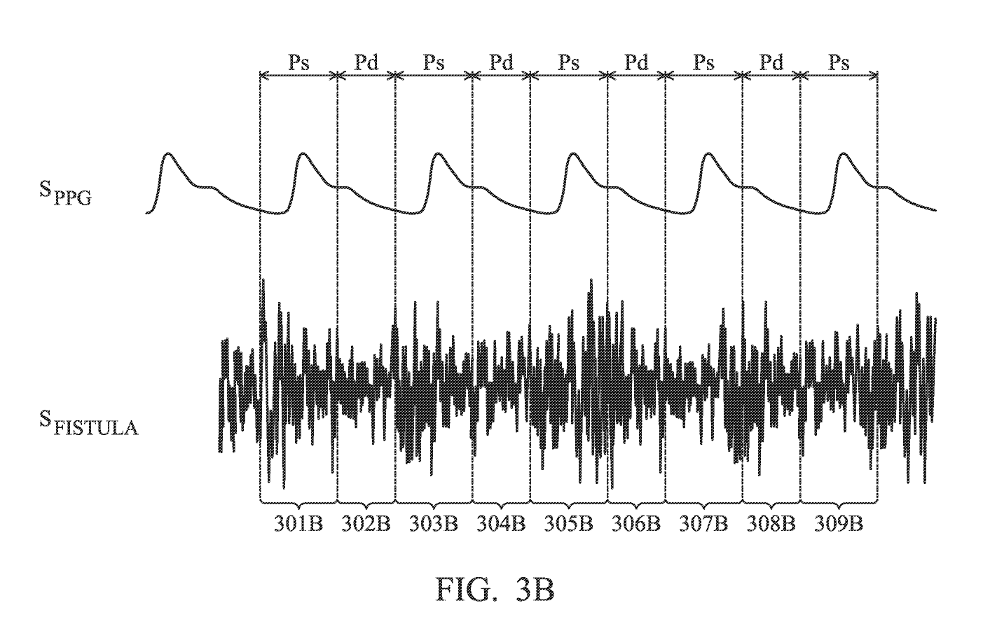

FIG. 3A shows an example illustrating a relationship between a physiological signal S_{ECG} and a sound signal S_{FISTULA} according to an embodiment of the invention. Referring to FIG. 1 and FIG. 3A together, the processing circuit 130 obtains the physiological signal S_{ECG} from the physiological signal sensor 110 and the sound signal S_{FISTULA} from the acoustic receiver 120, wherein the physiological signal S_{ECG} is an ECG lead signal. The processing circuit 130 analyzes the physiological signal S_{ECG} to obtain a diastolic phase Pd and a systolic phase Ps of some cardiac cycles of the user. Next, the processing circuit 130 divides the sound signal S_{FISTULA} into a plurality of intervals 301A-309A according to the diastolic phases Pd and the systolic phases Ps of the cardiac cycles. For example, each of the intervals 301A, 303A, 305A, 307A and 309A corresponds to the systolic phase Ps of the cardiac cycle, and each of the intervals 302A, 304A, 306A and 308A corresponds to the diastolic phase Pd of the cardiac cycle. Then, for the sound signal S_{FISTULA} corresponding to the intervals 301A, 303A, 305A, 307A and 309A, the processing circuit 130 uses a first algorithm to analyze the sound signal S_{FISTULA}. For the sound signal S_{FISTULA} corresponding to the intervals 302A, 304A, 306A and 308A, the processing circuit 130 uses a second algorithm to analyze the sound signal S_{FISTULA}. The sound signal S_{FISTULA} corresponding to the intervals 301A, 303A, 305A, 307A and 309A may be regarded as a first portion of the sound signal S_{FISTULA} and the sound signal S_{FISTULA} corresponding to the intervals 302A, 304A, 306A and 308A may be regarded as a second portion of the sound signal S_{FISTULA}. The processing circuit 130 may also just analyzes the first portion of the sound signal S_{FISTULA} but ignores the second portion of the sound signal S_{FISTULA} from analysis. Based on the analysis results, the processing circuit 130 may determine the degree of fistula stenosis.

FIG. 3B shows an example illustrating a relationship between a physiological signal S_{PPG} and a sound signal S_{FISTULA} according to an embodiment of the invention. Referring to FIG. 1 and FIG. 3B together, the processing circuit 130 obtains the physiological signal S_{PPG} from the physiological signal sensor 110 and the sound signal S_{FISTULA} from the acoustic receiver 120, wherein the physiological signal S_{PPG} is an PPG signal. Similarly, the processing circuit 130 can analyze the physiological signal S_{PPG} to obtain the diastolic phases Pd and the systolic phases Ps of the cardiac cycles of the user. Next, the processing circuit 130 divides the sound signal S_{FISTULA} into a plurality of intervals according to the diastolic phases Pd and the systolic phases Ps of the cardiac cycles. Then, the processing circuit 130 analyzes the sound signal S_{FISTULA} to obtain a degree of fistula stenosis of the user. For example, for the sound signal S_{FISTULA} corresponding to the intervals 301B, 303B, 305B, 307B and 309B, the processing circuit 130 uses a first algorithm to analyze the sound signal S_{FISTULA}. For the sound signal S_{FISTULA} corresponding to the intervals 302B, 304B, 306B and 308B, the processing circuit 130 uses a second algorithm to analyze the sound signal S_{FISTULA}. The sound signal S_{FISTULA} corresponding to the intervals 301B, 303B, 305B, 307B and 309B may be regarded as a first portion of the sound signal S_{FISTULA} and the sound signal S_{FISTULA} corresponding to the intervals 302B, 304B, 306B and 308B may be regarded as a second portion of the sound signal S_{FISTULA}. The processing circuit 130 may also just analyze the first portion of the sound signal S_{FISTULA} but ignores the second portion of the sound signal S_{FISTULA} from analysis. Based on the analysis results, the processing circuit 130 may determine the degree of fistula stenosis.

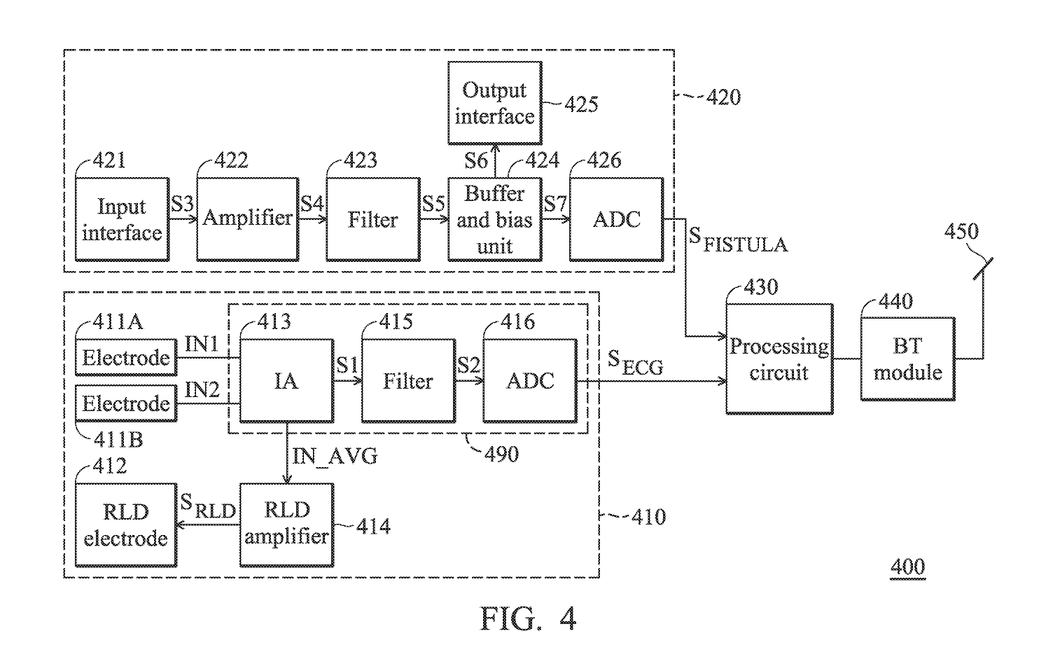

FIG. 4 shows a device 400 for detecting fistula stenosis of a user according to another embodiment of the invention. The device 400 comprises a physiological signal sensor 410, an acoustic receiver 420, a processing circuit 430, a bluetooth (BT) module 440, and an antenna 450. The physiological signal sensor 410 is an ECG signal sensor for providing a physiological signal S_{ECG}, and the physiological signal sensor 410 comprises two electrodes 411A and 411B, a right leg drive (RLD) electrode 412, a RLD amplifier 414 and an ECG lead signal generator 490, wherein RLD stands for right-leg drive. Note that the RLD electrode 412 and RLD amplifier 414 may be optional and may be removed for some appropriate situations. The ECG lead signal generator 490 comprises an instrumentation amplifier (IA) 413, a filter 415 and an analog to digital converter (ADC) 416. The IA 413 receives a skin voltage signal IN1 from the electrode 411A and a skin voltage signal IN2 from the electrode 411B when the device 400 contacts the user, and amplifies a difference between the skin voltage signals IN1 and IN2 to provide a signal S1. In the embodiment, the electrodes 411A and 411B are used to receive physiological signals from a first standard limb lead of the user. The first standard limb lead is from the user's right arm to left arm and is also called Lead-I signal. The potential difference between the electrodes 411A and 411B on the left and right arms can be measured by the physiological signal sensor 410. In one embodiment, the IA 413 further provides an average voltage IN_AVG of the skin voltage signals IN1 and IN2 to the RLD amplifier 414. The RLD amplifier 414 amplifies the average voltage IN_AVG to provide a signal S_{RLD} to the RLD electrode 412, wherein the signal S_{RLD} is a common mode voltage for the body of the user. Next, the filter 415 filters the signal S1 to provide a signal S2. The ADC 416 converts the signal S2 into the physiological signal S_{ECG}.

In FIG. 4, the acoustic receiver 420 functions as a stethoscope for collecting blood stream sound of, for example, a subcutaneous fistula of the user, to provide a sound signal S_{FISTULA}, and the acoustic receiver 420 comprises an input interface 421, an amplifier 422, a filter 423, a buffer and bias unit 424, an output interface 425, and an ADC 426. The input interface 421 may be a microphone, an auscultation head or a diaphragm, wherein the input interface 421 is capable of receiving blood-stream sound from the fistula of the user when contacting with the surface of the skin of the user, so as to provide a signal S3. In one embodiment, the microphone is a capacitive microphone with higher sensitivity and better frequency response, to obtain good sound quality. In another embodiment, the capacitive microphone may be disposed behind the diaphragm. The amplifier 422 amplifies the signal S3 to provide a signal S4. The filter 423 filters the signal S4 to provide a signal S5. The output interface 425 may be a speaker or an earphone jack for displaying the blood stream sound of the fistula. The buffer and bias unit 424 receives the signal S5 and provides a signal S6 to the output interface 425 according to the type of output interface 425. Furthermore, the buffer and bias unit 424 also provides a signal S7 according to the received signal S5. The ADC 426 converts the signal S7 into the sound signal S_{FISTULA}. After receiving the physiological signal S_{ECG} and the sound signal S_{FISTULA} the processing circuit 430 derives a degree of fistula stenosis according to the physiological signal S_{ECG} and the sound signal S_{FISTULA}. The processing circuit 430 obtains a physiological information according to the physiological signal S_{ECG}, and the processing circuit 430 distinguishes a first portion of the sound signal S_{FISTULA} and a second portion of the sound signal S_{FISTULA} according to the physiological information.

As described above, the physiological information may indicate the diastolic phase and/or the systolic phase of some cardiac cycles of the user, or indicate the arrhythmia duration and/or a non-arrhythmia duration of a plurality of cardiac cycles of the user. For instance, the first portion of the sound signal S_{FISTULA} may correspond to systolic phases of cardiac cycles of the user and the second portion of the sound signal S_{FISTULA} may correspond to diastolic phases of cardiac cycles of the user. Then, the processing circuit 430 analyzes at least one of the first portion of the sound signal to derive the degree of fistula stenosis. After obtaining the degree of fistula stenosis, the processing circuit 430 provides a result regarding the degree of fistula stenosis to a remote device via the BT module 440 and antenna 450. Furthermore, the processing circuit 430 also can provide the physiological signal S_{ECG} and the sound signal S_{FISTULA} to the remote device. The remote device may be a mobile device (such as a smartphone), a router (Hub) or a personal computer, etc., and the remote device is capable of transmitting the result regarding the degree of fistula stenosis, the physiological signal S_{ECG} and the sound signal S_{FISTULA} to various back-end services (e.g. applications of the mobile device, PC applications or cloud service) for signal processing and judgment.

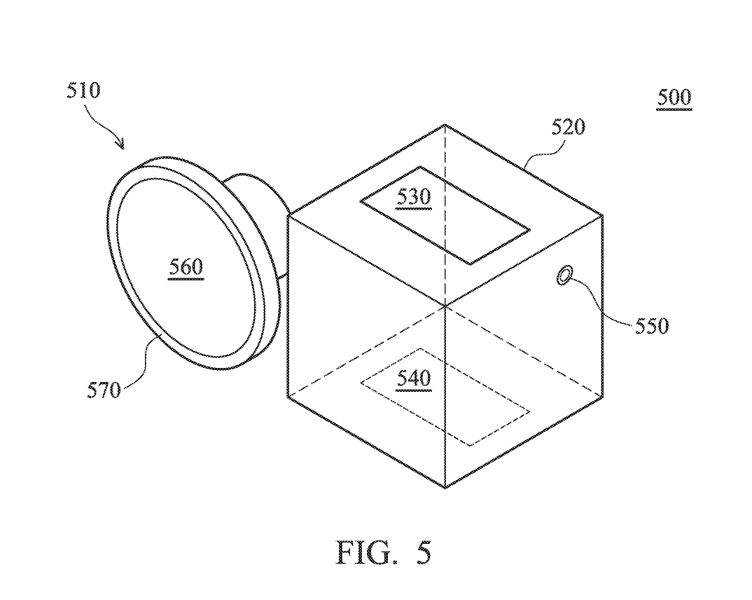

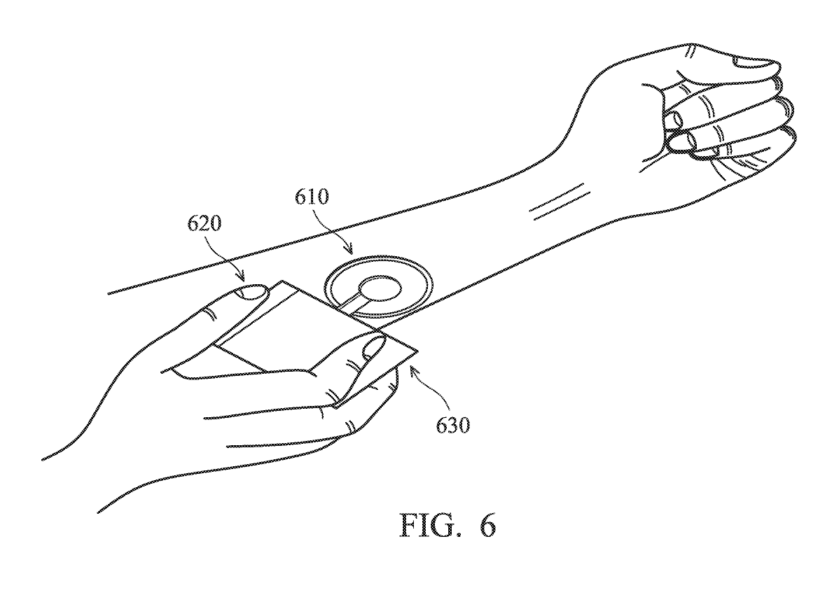

FIG. 5 shows an example model illustrating a device 500 for detecting fistula stenosis of a user according to an embodiment of the invention, and FIG. 6 shows a schematic illustrating using the device 500 of FIG. 5 to measure the fistula of the user. In FIG. 5, the device 500 comprises two components: an auscultation head 510 and a square case 520. The auscultation head 510 comprises a diaphragm 560 and an electrode ring 570. In the embodiment, the diaphragm 560 is an input interface (e.g. 421 of FIG. 4) for receiving blood stream sound from a fistula of the user when contacting with the surface of the skin of the user, e.g. the skin of the left arm of the user (as shown in label 610 of FIG. 6). In the embodiment, the fistula of the user may be an autogenous arteriovenous fistula or an arteriovenous fistula graft. Furthermore, the electrode ring 570 is an electrode (e.g. 411A of FIG. 4) for receiving a first skin voltage signal from the contacted surface of the skin of the user. The square case 520 comprises the electrodes 530 and 540 and an earphone jack 550. The electrode 530 (e.g. 411B of FIG. 4) is capable of receiving a second skin voltage signal from the skin of user's right arm (as shown in label 620 of FIG. 6). Furthermore, the electrode 540 (e.g. 412 of FIG. 4) is capable of providing a common mode voltage for the user (as shown in label 630 of FIG. 6). In the embodiment, the other circuits of the device 500 are implemented inside the square case 520, such as the amplifiers, filters, ADCs, processor, and BT module of FIG. 4 and so on. Thus, a processor of the device 500 obtains a sound signal S_{FISTULA} according to the blood stream sound, and also obtains a physiological signal S_{ECG} according to the first and second skin voltage signals, wherein the first and second skin voltage signals are obtained from a first standard limb lead or a Lead-I signal of the user. When the user holds the device 500, the right palm of the user can contact the electrodes 530 and 540 simultaneously. Furthermore, when the electrode ring 570 contacts the left arm of the user, a loop is formed by the electrodes 530 and 540 and the electrode ring 570, so as to obtain a stable single-lead ECG signal. For dialysis patients suffering from cardiac arrhythmias, the fistula blood stream sound will change in abnormal heart rhythm duration. The device 500 can determine the incidence of arrhythmias according to the physiological signal S_{ECG} and improve the accuracy of the algorithms in arrhythmia patients. For example, the processor of the device 500 may filter out the sound signal S_{FISTULA} corresponding to the arrhythmias duration, i.e. not to determine/analyze. In one embodiment, the processor of the device 500 may determine the type of the arrhythmias according to the physiological signal S_{ECG}, and use the algorithm corresponding to the type of the arrhythmias to analyze the sound signal S_{FISTULA}.

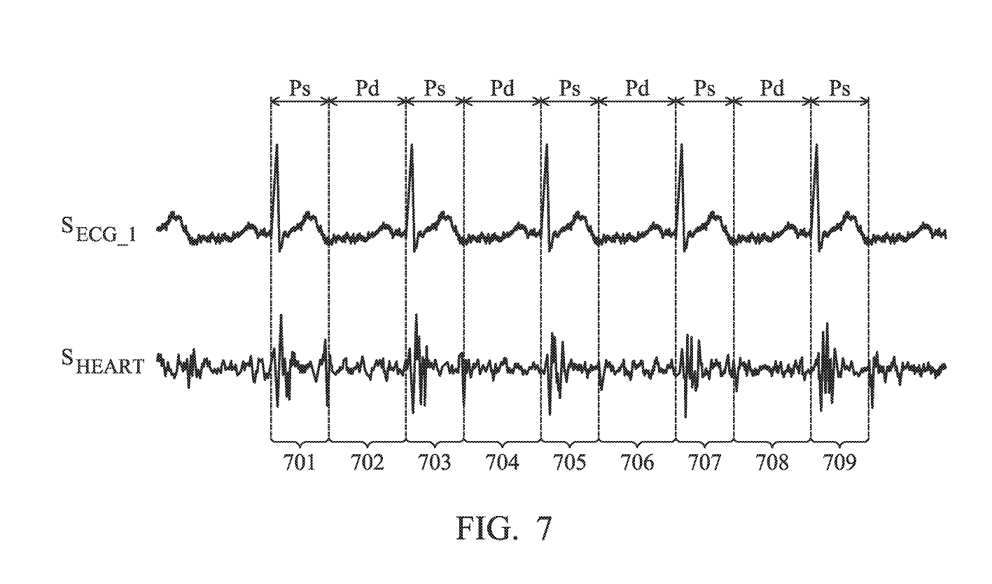

It is worth mentioning that the use of the device 500 of FIG. 5 is not limited to the scenario illustrated in FIG. 6. For instance, one change can be made to FIG. 6 by contacting the electrode ring 570 to skin around the heart of the user instead of contacting the electrode ring 570 on the skin of the left arm of the user. In this way, the diaphragm 560 may receive a heart sound signal S_{HEART} instead of a fistula sound signal S_{FISTULA}. Also, another ECG signal S_{ECG}_{_{—}}₁ may be provided according to the skin voltages acquired by electrode ring 570 and electrode 530. The electrode 540 may still provide a common mode voltage for the user. FIG. 7 shows an example illustrating a relationship between the another ECG signal S_{ECG}_{_{—}}₁ and the heart sound signal S_{HEART}. As it is already known that the heart sound signal S_{HEART} can be used to identify the diastolic and systolic phases of cardiac cycles, a plurality of intervals 701-709 can be marked according to the waveform of the heart sound signal S_{HEART}. In this example, each of the intervals 701, 703, 705, 707 and 709 corresponds to a systolic phase Ps of a cardiac cycle, and each of the intervals 702, 704, 706 and 708 corresponds to a diastolic phase Pd of a cardiac cycle. Then, by observing the S_{ECG}_{_{—}}₁ and the plurality of intervals 701-709, it can be confirmed the S_{ECG}_{_{—}}₁ bears a definite relationship with the diastolic and systolic phases of cardiac cycles. Therefore, in general, by analyzing ECG signal, it is able to obtain or identify intervals of systolic and diastolic phases of cardiac cycles.

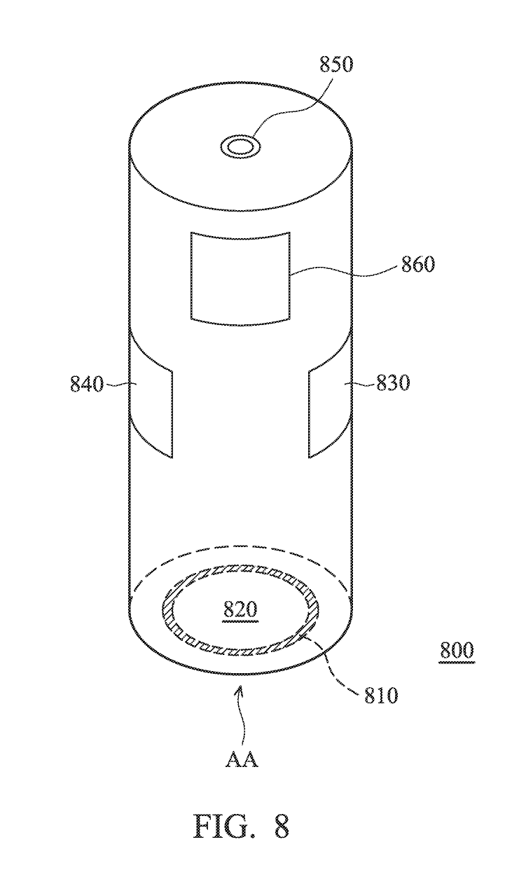

FIG. 8 shows an example model illustrating a device 800 for detecting fistula stenosis of a user according to another embodiment of the invention. The device 800 has a column architecture. The device 800 comprises an electrode ring 810, a microphone 820 surrounded by the electrode ring 810, an electrode 830, a RLD electrode 840, an earphone jack 850 and a display 860, wherein the electrode ring 810 and the microphone 820 are disposed in one side of the device 800 (as shown in label AA of FIG. 8). Unlike the device 500, the device 800 does not comprise the auscultation head 510. Furthermore, the display 860 of the device 800 is capable of displaying the result of the fistula stenosis for the user. To use the device 800, one can shed light from disclosure regarding FIG. 6. For example, a user may hold the device 800 with right palm to contact the electrode 830 and the RLD electrode 840 for acquiring a first skin voltage. Then, the user may further contact the electrode ring 810 and the microphone 820 on left arm to acquire a second skin voltage and a sound signal from the fistula of the user.

According to the embodiments, dialysis patients can perform home care to collect fistula blood sounds and physiological (e.g. ECG, PPG and so on) signals. Furthermore, information regarding the fistula blood sounds and the physiological signals can be transmitted to a remote device through a wireless transmission for interpretation. Moreover, the remote device can transmit the received information to the hospital or service center sent via a network for further analysis.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A device for detecting fistula stenosis, comprising:
a physiological signal sensor, configured for providing a physiological signal of a user;
an acoustic receiver, configured for detecting a sound from a fistula of the user to generate a sound signal; and
a processing circuit, configured for providing a degree of fistula stenosis according to the physiological signal of the user and the sound signal.

2. The device as claimed in claim 1, wherein the processing circuit comprises:
an information generator, deriving a physiological information according to the physiological signal;
a signal separator, distinguishing a first portion of the sound signal from a second portion of the sound signal according to the physiological information; and
a signal analyzer, analyzing at least one of the first portion of the sound signal and the second portion of the sound signal to derive the degree of fistula stenosis.

3. The device as claimed in claim 2, wherein the first portion of the sound signal corresponds to diastolic phases of cardiac cycles of the user and the second portion of the sound signal corresponds to systolic phases of cardiac cycles of the user.

4. The device as claimed in claim 2, wherein the first portion of the sound signal corresponds to an arrhythmia duration of the user and the second portion of the sound signal corresponds to a non-arrhythmia duration of the user.

5. The device as claimed in claim 1, wherein the physiological signal is an electrocardiogram (ECG) lead signal, and the physiological signal sensor comprises:
a first electrode, configured for detecting a first skin voltage from the user;
a second electrode, configured for detecting a second skin voltage from the user; and
an ECG lead signal generator coupled to the first electrode and the second electrode, configured for providing the ECG lead signal according to the first skin voltage and the second skin voltage.

6. The device as claimed in claim 5, wherein the first skin voltage and the sound from the fistula of the user are both provided from one arm of the user and the second skin voltage is provided from the other arm of the user.

7. A computing device for detecting fistula stenosis, comprising:
a processing circuit, comprising:
a processor, configured for providing a degree of fistula stenosis according to a physiological signal and a sound signal.

8. The computing device as claimed in claim 7, wherein the processor executes instructions for:
deriving a physiological information according to the physiological signal;
distinguishing a first portion of the sound signal from a second portion of the sound signal according to the physiological information; and
analyzing at least one of the first portion of the sound signal and the second portion of the sound signal to derive the degree of fistula stenosis.

9. The computing device as claimed in claim 8, wherein the first portion of the sound signal corresponds to diastolic phases of cardiac cycles of a user and the second portion of the sound signal corresponds to systolic phases of cardiac cycles of the user.

10. The computing device as claimed in claim 9, wherein the second portion of the sound signal is analyzed to derive the degree of fistula stenosis.

11. The computing device as claimed in claim 8, wherein the first portion of the sound signal corresponds to an arrhythmia duration of a user and the second portion of the sound signal corresponds to a non-arrhythmia duration of the user.

12. The computing device as claimed in claim 11, wherein the second portion of the sound signal is analyzed to derive the degree of fistula stenosis.

13. The computing device as claimed in claim 7, further comprising:
an output unit, configured for generating one of an audio signal and a visual signal according to the degree of fistula stenosis.

14. The computing device as claimed in claim 7, further comprising:
an acoustic receiver, configured for detecting a sound from a fistula of a user to generate the sound signal.

15. The computing device as claimed in claim 14, further comprising:
a physiological signal sensor, configured for providing the physiological signal from the user.

16. The computing device as claimed in claim 15, wherein the physiological signal is an electrocardiogram (ECG) lead signal, and the physiological signal sensor comprises:
a first electrode, configured for detecting a first skin voltage from the user;
a second electrode, configured for detecting a second skin voltage from the user; and
an ECG lead signal generator coupled to the first electrode and the second electrode, configured for providing the ECG lead signal according to the first skin voltage and the second skin voltage.

17. The computing device as claimed in claim 16, wherein the first skin voltage and the sound from the fistula of the user are both provided from one arm of the user and the second skin voltage is provided from the other arm of the user.

18. A method for detecting fistula stenosis, comprising:
at a computing device,
receiving a physiological signal of a user, wherein the physiological signal is provided by a physiological signal sensor;
receiving a sound signal generated by an acoustic receiver detecting a sound from a fistula of the user; and
providing a degree of fistula stenosis according to the physiological signal of the user and the sound signal.

19. The method as claimed in claim 18, wherein providing the degree of fistula stenosis comprises:
deriving a physiological information according to the physiological signal;
distinguishing a first portion of the sound signal from a second portion of the sound signal according to the physiological information; and
analyzing at least one of the first portion of the sound signal and the second portion of the sound signal to derive the degree of fistula stenosis.

20. The method as claimed in claim 19, wherein the first portion of the sound signal corresponds to diastolic phases of cardiac cycles of the user and the second portion of the sound signal corresponds to systolic phases of cardiac cycles of the user.

21. The method as claimed in claim 19, wherein the first portion of the sound signal corresponds to an arrhythmia duration of the user and the second portion of the sound signal corresponds to a non-arrhythmia duration of the user.

22. The method as claimed in claim 19, wherein the physiological signal is an electrocardiogram (ECG) lead signal, and the physiological signal sensor comprises:
a first electrode, configured for detecting a first skin voltage from the user;
a second electrode, configured for detecting a second skin voltage from the user; and
an ECG lead signal generator coupled to the first electrode and the second electrode, configured for providing the ECG lead signal according to the first skin voltage and the second skin voltage.

23. The method as claimed in claim 22, wherein the first skin voltage and the sound are both provided from one arm of the user and the second skin voltage is provided from the other arm of the user.

24. A computer readable storage medium having stored therein instructions, which when executed by a device, cause the device to:
provide a degree of fistula stenosis according to a physiological signal of a user and a sound signal.

25. The computer readable storage medium as claimed in claim 24, wherein providing the degree of fistula stenosis comprises:
deriving a physiological information according to the physiological signal of the user;
distinguishing a first portion of the sound signal from a second portion of the sound signal according to the physiological information; and
analyzing at least one of the first portion of the sound signal and the second portion of the sound signal to derive the degree of fistula stenosis.

26. The computer readable storage medium as claimed in claim 25, wherein the first portion of the sound signal corresponds to diastolic phases of cardiac cycles of the user and the second portion of the sound signal corresponds to systolic phases of cardiac cycles of the user.
